(19) United States
(12) Patent Application Publication
(10) Pub. No.: US 2005/0112084 A1
(43) Pub. Date: May 26, 2005

(54) Topical cosmetic composition

(75) Inventors: Janet K. O'Grady, Westwood, MA (US); Maureen Sullivan Morrissey, Belmont, MA (US); Alexander Novikov, Framingham, MA (US); Ronald McLaughlin, Medford, MA (US)

Correspondence Address: PATENT AND TRADEMARK COUNSEL;THE GILLETTE COMPANY, 800 BOYLSTON STREET, BOSTON, MA, 02199, US

(73) Assignee: The Gillette Company

(21) Appl. No.: 10/719,224

(22) Filed: Nov. 21, 2003

(51) Int. Cl. A61K 7/15; A61K 7/06; A61K 31/525; A61K 31/355; A61K 35/78

(52) U.S. Cl. 424/73; 424/74; 424/744; 514/458; 514/474; 514/251; 514/276

(57) ABSTRACT

The present invention embraces a topical cosmetic composition for providing noticeably healthy looking human skin. The composition comprises a topical cosmetic carrier vehicle including vitamin A, vitamin E, aloe, and at least one member selected from the group consisting of bisabolol, panthenol, vitamin B₃ and vitamin C. Preferably, the composition includes vitamin B₃. More preferably, the composition also includes vitamin C and/or bisabolol, most preferably both of these. The present invention also embraces a method of providing noticeably healthy looking human skin which comprises applying to human skin at least once a day over at least seven days a topical cosmetic composition as described above.

### BACKGROUND OF THE INVENTION

The present invention relates to a topical cosmetic composition for providing noticeably healthy looking human skin.

Topical skin treating compositions containing a vitamin B₃ compound and other active agents are disclosed in U.S. Pat. No. 6,217,888 and U.S. Pat. No. 6,238,678. These compositions are used to regulate skin condition such as, for example, tactile discontinuities, wrinkles, pore size, flakiness, roughness, elasticity, discoloration, spider vessels, etc. The patents suggest that a wide variety of other optional cosmetic ingredients may be included in the skin treating compositions including other vitamin components. U.S. 2003/0026775 describes self-foaming shaving lotions that may include optional cosmetic ingredients including, among other things, various skin conditioning agents such as vitamins, amino acids, aloe, allantoin, botanical oils, etc. U.S. Pat. No. 6,544,534 discloses a skin conditioner that includes aloe and vitamin E, among other things, to provide “skin like an angel.”

### SUMMARY OF THE INVENTION

The present invention embraces a topical cosmetic composition for providing noticeably healthy looking human skin. The composition comprises a topical cosmetic carrier vehicle including vitamin A, vitamin E, aloe, and at least one member (preferably at least two members) selected from the group consisting of bisabolol, panthenol, vitamin B₃ and vitamin C. Preferably, the composition includes vitamin B₃. More preferably, the composition also includes vitamin C and/or bisabolol, most preferably both of these. The present invention also embraces a method of providing noticeably healthy looking human skin which comprises applying to human skin at least once a day over at least seven days a topical cosmetic composition as described above.

### DETAILED DESCRIPTION OF THE INVENTION

The topical cosmetic composition of the present invention may be in the form of a liquid, lotion, cream, gel (including non-aerosol, aerosol, and self-foaming gel), foam, mousse, spray (aerosol or pump), paste or solid (e.g., stick or bar). It may be in the form of a solution, such as an aqueous or alcoholic or hydroalcoholic solution, or an emulsion, including both a water-in-oil emulsion and an oil-in-water emulsion. A preferred composition is an oil-in-water emulsion, particularly an oil-in-water emulsion that comprises at least 70% water by weight of the composition. Accordingly, the composition will include a dermatologically acceptable topical cosmetic carrier vehicle into which the various active ingredients are dissolved or dispersed.

Without being limited thereto, typical materials which may be included in the cosmetic carrier vehicle are as follows: water; lower monovalent alcohols (e.g., C₁-C₄, particularly ethanol); polyhydric alcohols having 3 to 6 carbon atoms and 2 to 6 hydroxyl groups (e.g., propylene glycol, glycerin, sorbitol, butanediol, hexanetriol, etc.); silicones, including volatile silicones (e.g., cyclomethicone) and non-volatile silicones (e.g., dimethicone or phenyl trimethicone); surfactants, including anionic, cationic, non-ionic and amphoteric surfactants; cleansing or lathering agents (e.g., soaps and interrupted soaps, such as the sodium, potassium and lower alkanolamine salts of C₁₀-C₂₀ fatty acids and N-fatty (C₁₀-C₂₀) acyl sarcosines); gelling or thickening agents (e.g., hydroxyalkyl cellulose, carboxymethyl cellulose, cellulose methyl ether, Carbopols, gums); non-volatile paraffinic hydrocarbon fluids (e.g., mineral oil, hydrogenated polyisobutene); fatty alcohols (e.g., myristyl alcohol, stearyl alcohol, octyl dodecanol); fatty acid esters or fatty alcohol esters (e.g., isopropyl myristate and C₁₂-₁₅ alcohols benzoate); dicarboxylic acid esters (e.g., diisopropyl sebacate); fatty amides (e.g., Stearamide MEA); ethoxylated and/or propoxylated ethers and esters of C₄-C₂₀ alcohols and acids (e.g., PPG-5-Buteth-7 and PEG-8 Distearate); propellants or foaming agents or self-foaming agents (e.g., volatile hydrocarbons with a vapor pressure of about 3 to about 70 psig at 20° C.); emollients; humectants; suspending agents; fillers; emulsifiers; fragrances, colorants; uv-absorbing agents or sunscreen agents; antimicrobial agents; preservatives; etc.

The foregoing list of materials is by way of example only and is not intended to be a comprehensive list of all potential materials that may be useful in a topical cosmetic composition. Obviously, the skilled worker may select appropriate materials that will provide the desired application and aesthetic characteristics and attributes of the particular cosmetic composition to be produced. Typical cosmetic compositions may be formulated as a non-aerosol shaving gel, an aerosol self-foaming shaving gel, an aftershave lotion, an aftershave gel, a moisturizing lotion or cream, a cleansing wash, and a soap bar.

In addition to the topical cosmetic carrier vehicle, the composition will include the various active ingredients, namely vitamin A, vitamin E, aloe, and at least one member (preferably at least two members) selected from the group consisting of bisabolol, panthenol, vitamin B₃ and vitamin C. Each of the aforementioned ingredients independently may be included in an amount of about 0.001% to about 5%, preferably about 0.01% to about 2%, more preferably about 0.01% to about 1%, by weight. While each of the above ingredients may be added separately and independently, in some cases it may be advantageous to add a group of certain of the above ingredients as a mixture, for example where such a mixture is commercially available (e.g., a liposome mixture of vitamins A, C and E).

By vitamin A is meant vitamin A and any of its natural or synthetic analogs or derivatives that have vitamin A biologic activity. These include retinol, retinol esters (e.g., retinyl palmitate, retinyl acetate and retinyl propionate), retinal, retinoic acid and retinoic acid esters (e.g., tocopheryl retinoate).

By vitamin B₃ is meant vitamin B₃ and any of its natural or synthetic analogs or derivatives that have vitamin B₃ biologic activity. These include niacinamide, nicotinic acid, nicotinyl alcohol and derivatives (e.g., esters such as tocopheryl nicotinate and nicotinyl acetate) and salts thereof. A thorough description of useful vitamin B₃ compounds is provided in U.S. Pat. No. 6,238,678, which is incorporated herein by reference.

By vitamin C is meant vitamin C and any of its natural or synthetic analogs or derivatives that have vitamin C biologic activity. These include ascorbic acid and salts and esters thereof such as sodium ascorbate and ascorbyl palmitate.

By vitamin E is meant vitamin E and any of its natural or synthetic analogs or derivatives that have vitamin E biologic activity. These include tocopherol and esters thereof, such as tocopheryl acetate and tocopheryl sorbate.

By panthenol is meant panthenol and any of its analogs or derivatives that have panthenol biologic activity. These include panthenol, its ether and ester derivatives such as panthenol ethyl ether and panthenyl triacetate, and pantothenic acid. Panthenol is preferred. Panthenol is also sometimes referred to as vitamin B₅.

Bisabolol may be synthetically produced or derived from chamomile and is commercially available from a variety of sources.

Aloe is commercially available from a variety of sources that manufacture bulk supplies of the aloe in liquid, gel or dry (e.g., lyophilized) form.

The present invention also embraces a method of providing noticeably healthy looking human skin which comprises applying to human skin at least once a day over at least seven days, preferably at least fourteen days, a topical cosmetic composition as described above. Ideally, a topical cosmetic composition in accordance with the present invention should be applied twice a day over at least fourteen days to achieve the most beneficial effects. Of course, one could apply two different compositions within the scope of the present invention (e.g., a shaving gel and an aftershave lotion) during each day rather than two applications of the same composition. Noticeably healthy looking skin will typically be evidenced by five signs or attributes, namely clean and clear, non-irritated, moisturized, smooth and even-toned.

The invention may be further described by the following examples in which all parts and percentages are by weight. Each of the formulations set out in the examples below are made in a conventional manner.

### Example 1

Aerosol Shave Gel

| Ingredient | Wt. % |
|---|---|
| Water | 73.31 |
| Stearoyl sarcosine (and) | 6.73 |
| Myristoyl sarcosine | |
| Laureth-23 | 6.06 |
| Myristyl alcohol | 2.79 |
| Triethanolamine | 2.45 |
| Laureth-4 | 1.73 |
| Glycerin | 0.96 |
| Mineral oil | 0.87 |
| PEG-10 Soya sterol | 0.24 |
| PEG-23M | 0.24 |
| Polyquaternium-10 | 0.24 |
| Hydroxyethyl cellulose | 0.10 |
| Vitamin E acetate | 0.10 |
| Vitamin B₃ | 0.10 |
| Vitamin A palmitate | 0.01 |
| Aloe leaf juice | 0.01 |
| Bisabolol | 0.01 |
| Preserv/Fragr/Color/Other | 0.20 |
| Isobutane/Isopentane | 3.85 |

### Example 2

Non-Aerosol Shave Gel

| Ingredient | Wt. % |
|---|---|
| Water | 81.87 |
| Sodium laureth sulfate | 6.00 |
| Cocamidopropyl betaine | 4.00 |
| Glycerin | 3.00 |
| Acrylates C₁₀₋₃₀ alkyl acrylates | 1.50 |
| Coceth-7 & PPG-1-PEG Lauryl Glycol | 1.00 |
| Ether & PEG-40 Hydrog. Castor Oil | |
| Sodium hydroxide (50%) | 0.72 |
| Mineral oil | 0.25 |
| Polyquaternium-10 | 0.25 |
| Panthenol | 0.20 |
| Liposome vitamin A/C/E | 0.20 |
| Vitamin E acetate | 0.20 |
| Vitamin B₃ | 0.20 |
| Bisabolol | 0.15 |
| Aloe leaf juice | 0.01 |
| Preserv/Fragr/Color/Other | 0.45 |

### Example 3

After-Shave Gel

| Ingredient | Wt. % |
|---|---|
| Water | 83.52 |
| Ethanol | 10.81 |
| PEG-12 Dimethicone | 2.00 |
| PPG-26 Buteth-26 (and) | 0.75 |
| PEG-40 Hydrog. Castor Oil | |
| Triethanolamine | 0.62 |
| Phenoxyethanol | 0.50 |
| Carbomer | 0.45 |
| Menthyl lactate | 0.25 |
| PEG-20 Glyceryl Laurate | 0.25 |
| Liposome vitamin A/C/E | 0.20 |
| Vitamin B₃ | 0.20 |
| Bisabolol | 0.15 |
| Polysorbate 80 | 0.15 |
| Aloe leaf juice | 0.01 |
| Preserv/Fragr/Color/Other | 0.14 |

### Example 4

Moisturizing Lotion

| Ingredient | Wt. % |
|---|---|
| Water | 77.64 |
| Octinoxate | 5.00 |
| Cyclomethicone | 3.00 |
| Isononyl Isonanoate | 3.00 |
| Oxybenzone | 2.00 |
| Behenyl alcohol & Stearyl alcohol (&) | 2.50 |
| Arachidyl alcohol & Behenyl alcohol | |
| Polyacrylamide & C₁₃₋₁₄ Isoparaffin | 1.40 |
| & Laureth-7 | |
| PEG-20 Glyceryl Laurate | 1.80 |
| Glyceryl monostearate | 1.00 |
| Dimethicone | 0.75 |
| Cholesterol | 0.70 |
| Lecithin | 0.20 |
| Liposome vitamin A/C/E | 0.20 |
| Vitamin B₃ | 0.20 |
| Bisabolol | 0.15 |
| Vitamin E acetate | 0.10 |
| Aloe leaf juice | 0.01 |
| Preserv/Fragr/Color/Other | 0.35 |

### Example 5

Cleansing Wash

| Ingredient | Wt. % |
|---|---|
| Water | 74.30 |
| Acrylates copolymer | 12.00 |
| Sodium laureth sulfate | 8.00 |
| Glycerin | 3.00 |
| Sodium methyl cocoyl taurate | 1.00 |
| Decyl glucoside | 0.50 |
| Sodium hydroxide | 0.50 |
| Triclosan | 0.10 |
| Panthenol | 0.05 |
| Vitamin B₃ | 0.02 |
| Aloe leaf juice | 0.01 |
| Vitamin A palmitate | 0.001 |
| Vitamin E acetate | 0.001 |
| Preserv/Fragr/Color/Other | 0.518 |

### Example 6

Cleansing Bar

| Ingredient | Wt. % |
|---|---|
| Sodium palmate | 58.17 |
| Sodium cocoate/sodium palmkernelate | 14.54 |
| Water | 14.00 |
| Glycerin | 4.67 |
| Sorbitol | 2.20 |
| Sodium dilinoleate | 1.97 |
| Stearic acid | 1.81 |
| Propylene glycol | 0.99 |
| Potassium palmate | 0.75 |
| Potassium cocoate | 0.19 |
| Triclosan | 0.15 |
| Bisabolol | 0.15 |
| Aloe leaf juice | 0.12 |
| Potasssium stearate | 0.06 |
| Essential oil blend | 0.05 |
| Vitamin B₃ | 0.02 |
| Vitamin A palmitate | 0.01 |
| Vitamin C | 0.01 |
| Vitamin E acetate | 0.01 |
| Preserv/Color/Other | 0.13 |

While particular embodiments of the invention have been shown and described for illustrative purposes, it will be apparent to those skilled in the art that various changes and modifications may be made without departing from the spirit and scope of the invention, which is defined by the claims which follow.

1. A topical cosmetic composition for providing noticeably healthy looking human skin comprising a topical cosmetic carrier vehicle including vitamin A, vitamin E, aloe, and at least one member selected from the group consisting of bisabolol, panthenol, vitamin B₃ and vitamin C, wherein said vitamin A, said vitamin E, said aloe, and said at least one member are each independently included in an amount of about 0.001% to about 5% by weight of the composition.

2. The composition of claim 1 comprising at least two members selected from the group consisting of bisabolol, panthenol, vitamin B₃ and vitamin C.

3. The composition of claim 1 comprising vitamin B₃.

4. The composition of claim 1 or 3 comprising vitamin C.

5. The composition of claim 2 comprising bisabolol and vitamin B₃.

6. The composition of claim 5 comprising vitamin C.

7. The composition of claim 2 or 6 in the form of a liquid, lotion, cream, gel, foam, mousse, spray, paste or solid.

8. The composition of claim 2 in the form of a shaving gel.

9. The composition of claim 2 in the form of an aftershave gel or lotion.

10. The composition of claim 2 in the form of a cleansing wash, a cleansing bar or a moisturizing-lotion.

11. A method of providing noticeably healthy looking human skin which comprises applying to human skin at least once a day over at least seven days a topical cosmetic composition comprising a topical cosmetic carrier vehicle including vitamin A, vitamin E, aloe, and at least one member selected from the group consisting of bisabolol, panthenol, vitamin B₃ and vitamin C, wherein said vitamin A, said vitamin E, said aloe, and said at least one member are each independently included in an amount of about 0.001% to about 5% by weight of the composition.

12. The method of claim 11 wherein the topical cosmetic composition comprises at least two members selected from the group consisting of bisabolol, panthenol, vitamin B₃ and vitamin C.

13. The method of claim 11 wherein the topical cosmetic composition comprises vitamin B₃.

14. The method of claim 11 or 13 wherein the topical cosmetic composition comprises vitamin C.

15. The method of claim 12 wherein the topical cosmetic composition comprises bisabolol and vitamin B₃.

16. The method of claim 15 wherein the topical cosmetic composition comprises vitamin C.

17. The method of claim 12 or 16 wherein the topical cosmetic composition is in the form of a liquid, lotion, cream, gel, foam, mousse, spray, paste or solid.

18. The method of claim 12 wherein the topical cosmetic composition is in the form of a shaving gel.

19. The method of claim 12 wherein the topical cosmetic composition is in the form of an aftershave gel or lotion.

20. The method of claim 12 wherein the topical cosmetic composition is in the form of a cleansing wash, a cleansing bar or a moisturizing lotion.

21. The method of claim 11 or 12 which comprises applying the topical cosmetic composition to human skin at least twice a day over at least fourteen days.
